(19) United States
(12) Patent Application Publication
(10) Pub. No.: US 2019/0374643 A1
(43) Pub. Date: Dec. 12, 2019

(54) The Larger Size of Human White Blood Cells Compared to Red Blood Cells and its Effects on Development and Treatment of Cancers and Chronic Diseases

(71) Applicant: Iman Rezanezhad Gatabi, Grand Forks, ND (US)

(72) Inventor: Iman Rezanezhad Gatabi, Grand Forks, ND (US)

(21) Appl. No.: 16/541,086

(22) Filed: Aug. 14, 2019

(51) Int. Cl. A61K 45/06; A61N 5/10

(52) U.S. Cl. CPC A61K 45/06; A61N 2005/1087; A61N 5/10

(57) ABSTRACT

This application discloses methods for prevention and treatment of cancers and chronic diseases. Due to the larger size of human white blood cells compared to red blood cells, a decrease in the diameter of a part of a blood vessel can block the flow of white blood cells while allowing the flow of red blood cells. As a result, in tissues with such reduction in the diameter of blood vessels, there are areas where red blood cells are present, while there is a lack of white blood cells to fight the parasites and cancer cells. Therefore, cancer cells and other parasites can reside in such areas, replicate, and develop into diseases.

The disclosed methods provide for widening of the blood vessels in which a reduction in the diameter of a part of the vessel blocks the flow of white blood cells, while it allows the flow of red blood cells. As a result of this widening, white blood cells may pass through the vessel, reach to the parasites or cancer cells, and fight them. The disclosed methods include administration of vasodilators, blood fat-reducing drugs, or blood clot-reducing medications. The claimed methods may be used in combination with other conventional methods for treating cancer or chronic diseases.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The root causes of development of many chronic diseases and cancers in humans are unknown. Literature shows that intermittent fasting can be effective in treatment of many of chronic diseases. See Ref [1]. Researchers also proved that administration of anti-inflammatory drugs such as Aspirin can reduce the mutation rate of certain cancer cells. In addition, researches also demonstrated that fat people are more likely to develop certain types of cancers. However, the real reasons for occurring the aforementioned events are unknown. It is also unknown that why certain animals such as elephants, sharks, wales, and naked mole rates very rarely or never develop cancers. See references [2]-[4].

Many infectious diseases (such as Lyme disease and Brucellosis) may become chronic. The bacteria and viruses associated with those diseases can remain dormant in body tissues for years and cause a disease relapse in future. Some bacteria associated with chronic diseases are able to create a shield around themselves known as biofilms to protect themselves from antibiotics while they are in the dormant state. However, it is unknown why the said bacteria and viruses are able to stay dormant in parts of body tissues and live for years.

This application discloses that, among other things, the larger sizes of human White Blood Cells (WBCs) compared to Red Blood Cells (RBCs) are the cause of development of many types of chronic diseases and cancers. In areas of the tissue where the diameter of blood vessels are small enough to block the WBCs, and large enough to allow the RBCs to pass, cancer cells, viruses, and bacteria are more likely to develop, replicate, or remain dormant. Due to the presence of RBCs in those areas, bacteria, viruses, fungi, and cancer cells are provided with nutrition and oxygen while there is a lack of WBCs to fight the said bacteria, viruses, or cancer cells. Unlike humans, animals such as elephants and wales that very rarely or never develop cancers, have lymphocytes (a type of WBC that fights cancer) that are smaller than their RBCs. Therefore, there is no region in the tissue of elephants or wales that RBCs can be present without the presence of the said lymphocytes. As a result, lymphocytes (that are present along with RBCs) fight the cancer cells that tend to mutate and develop into tumors in elephants and wales.

This application claims that methods that in any way widen the diameter of the blood vessels may be effective for treatment or prevention of many chronic diseases and cancers. Such methods include body fat loss through fasting, administration of vessel widening medications known as vasodilators, administration of triglycerides lowering medication to decrease the vessel clogging, administration of medications that decrease blood clogging, and body work out and exercise to widen the blood vessels. Said methods may be used separately or in combination of each other. They may also be used in combination of other existing methods such as radiotherapy, chemotherapy, and administration of antibiotic and antiviral medications.

### BRIEF SUMMARY OF THE INVENTION

This application discloses methods for prevention and treatment of many types of cancers and chronic diseases. The disclosed methods are developed based on a theory that, among other things, the larger size of human WBCs compared to their RBCs are the root cause of development of many chronic diseases and cancers. A decrease in the diameter of a part of a blood vessel can create “safe zones” in human tissues for development and replication of cancer cells, bacteria, fungi, and viruses. The diameters of the blood vessels that supply blood to the said “safe zones” are large enough to allow the red blood cells to pass and enter the “safe zones” and small enough to block at least some types of white blood cells. As a result, cancer cells, bacteria, or viruses that reside in the “safe zones” are provided with oxygen and nutrition, while there is a lack of blocked white blood cells in the safe zones to fight the said cancer cells, bacteria, fungi, or viruses. The decreased diameters of the vessels that supply blood to the “safe zones” may be a result of inflammation, high body fat ratio, fat clogging in vessels, or blood clogging in the vessels. It may also be due to the natural small diameters of vessels that supplies blood to the said “safe zones.”

The theory provided herein also explains why some animals like elephants or wales very rarely or never develop cancers. Unlike humans, wales and elephants have lymphocytes (a type of WBC that fights cancers) that are smaller than RBCs. As a result, there will not be any place in the tissue of wales or elephants where RBCs can exist without the existence of the said lymphocytes. Therefore, the said “safe zones” for development and mutation of cancer cells do not exist in wales and elephants.

The claimed invention herein for treatment of cancers and chronic diseases comprises of methods to increase the diameters of blood vessels that supply blood to the “safe zones.” As a result, WBCs are able to enter the safe zones and fight cancer cells or infections. Applying the claimed methods to a healthy person may prevent the disease in the first place. Such methods include body fat loss through fasting, administration of vessel widening medications known as vasodilators, administration of triglycerides lowering medications to decrease the vessel clogging, administration of medications that decrease the blood clogging, and body work out and exercise to widen the blood vessels. Methods that decrease the size of WBCs in blood may also be effective. Said methods may be used separately or in combination of each other. They may also be used in combination with other existing methods such as radiotherapy, chemotherapy, and administration of antibiotic, antifungal, or antiviral medications.

### BRIEF DESCRIPTION OF THE SEVERAL VIEWS OF THE DRAWING

FIG. 1 illustrates a blood vessel which is partly clogged by fat. The opening in the fat is large enough to allow the RBCs to pass from left side of the vessel to the right side. The opening in the fat is small enough so that it blocks the WBCs. Therefore, no WBC is present at the right side of the vessel which allows for formation of a safe zone for bacteria, fungi, viruses, cancer cells, and fungi to grow and replicate.

FIG. 2 shows a table that compares the size of RBCs and lymphocytes in human, Asian elephants, and whale shark Rhincodon typus.

### DETAILED DESCRIPTION OF THE INVENTION

There are five main types of white blood cells in human body: Neutrophils, Eosinophils, Basophils, Lymphocytes, and Monocytes. Neutrophils mainly target bacteria and fungi. Eosinophils are responsible for fighting multicellular parasites and certain infections in vertebrates. Basophils are responsible for releasing histamine for inflammatory responses. Lymphocytes have three main subtypes: B cells, T cells, and Natural killer cells. Natural killer cells, CD8 and cytotoxic T cells target virus-infected and tumor cells. Monocytes can differentiate into macrophages and myeloid lineage dendritic cells. They also influence the process of adaptive immunity.

The diameters of Neutrophils, Eosinophils, Basophils, small Lymphocytes, large Lymphocutes, and Monocytes in humans are 12-15 um, 12-15 um, 12-15 um, 7-10 um, 14-20 um, and 15-30 um respectively [5]. However, RBCs have a diameter of 6-8 um. The diameter of the human blood vessels varies enormously from 25 mm in the aorta to 8 um in the capillaries. Therefore, even if there is no vessel clogging, in areas that the diameters of the blood vessels are small (8 um for example) may types of WBCs may not be present. However, RBCs are present in such areas. In addition, the presence of RBCs with absence of some types of WBCs may occur in vessels with larger natural diameters where the diameter of a part of vessel is partly decreased due to inflammation, fat clogging, or pressure from adjacent tissues. For example, if the diameter of a blood vessel is partly decreased to less than 7 um due to fat clogging, RBCs with diameters of less than 7 um may pass and all types of WBCs are blocked. If for example, the diameter of blood vessel is decreased down to 9 um, small lymphocytes and RBCs are able to pass and all other types of WBCs are blocked. Such presence of RBCs in absence of WBCs creates “safe zones” for cancer cells to mutate and replicate, and for viruses, fungi and bacteria to reside, replicate, or stay dormant. Depending on the type of the WBC that is blocked, the “safe zone” may be an appropriate area for growth and replication of cancer cells, viruses, bacteria, or fungi, or a combination of them or all of them. The presence of oxygen and nutrition in “safe zones” in the absence of WBCs provide a safe haven for cancer cells, bacteria, viruses, and fungi to reside and replicate and develop into diseases.

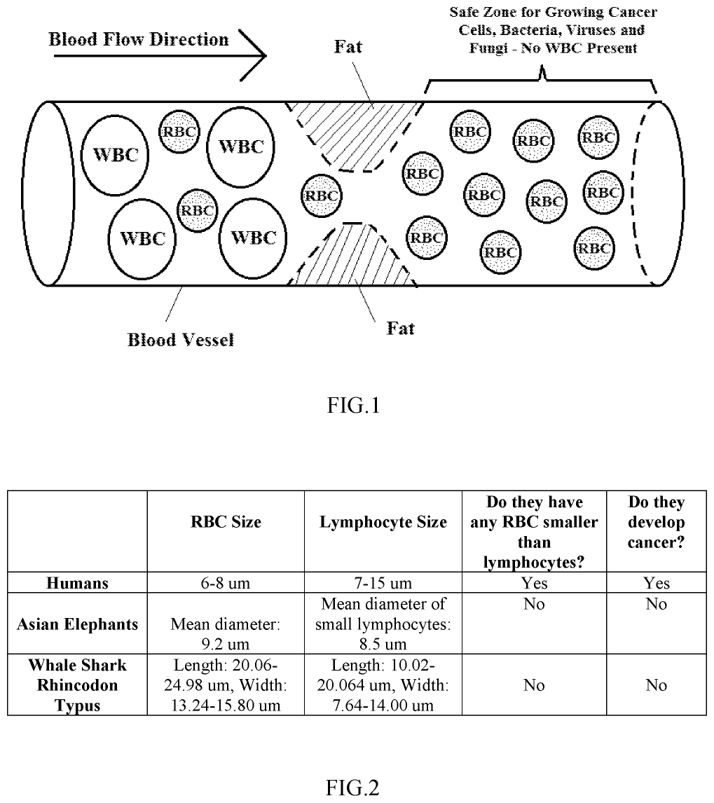

FIG. 1 illustrates a blood vessel which is partly clogged by fat. The opening in the fat is large enough to allow the RBCs to pass from left side of the vessel to the right side. The opening in the fat is small enough so that it blocks the WBCs. Therefore, no WBC is present at the right side of the vessel which allows for formation of a safe zone for bacteria, fungi, viruses, cancer cells, and fungi to grow and replicate.

The bacteria, viruses, fungi, or other parasites with smaller sizes, particularly those smaller than RBCs, are more likely to path through openings in fat or vessels with smaller diameters into the “safe zones” and develop chronic diseases or tumors. Likewise, cancer cells with smaller sizes are more likely to enter the “safe zones” through the blood flow and metastasize. For example, Borrelia burgdorferi, a bacteria that causes Lyme disease is a worm-shape bacteria which is 1 um wide and up to 10-25 um long. Due to its worm-like shape, it can pass through openings that RBCs are able to pass and enter the “safe zones.” As another example, Brucella which is the bacteria responsible for development of Brucellosis, is 0.6-1.5 um long and 0.5-0.7 um wide. The size of Brucella is much smaller than RBCs and therefore it is able to pass into the “safe zones” along with RBCs and develop chronic disease.

Many animals such as elephants, wales, sharks, and naked mole rates very rarely or never develop cancers. See references [2]-[4]. The diameters of the blood cells of Asian elephants and whale shark Rhincodon typus are reported in the literature. See references [6] and [7]. Unlike humans, both Asian elephants and whale shark Rhincodon typus have lymphocytes that are smaller than their RBCs. The lymphocytes of whale shark Rhincodon typus have a length of 10.02-20.064 um and a width of 7.64-14.00 um. The RBCs of whale shark Rhincodon typus have a length of 20.06-24.98 um and a width of 13.24-15.80 um [6]. The mean diameter of small lymphocytes in Asian elephants is 8.5 um while the mean diameter of RBCs is 9.2 um [7]. The smaller size of lymphocytes in Asian elephants and whale shark Rhincodon typus compared to their RBC size allows those small lymphocytes to pass throw narrow blood vessels that RBCs are able to pass. Therefore, there will not be any area in blood vessels of Asian elephants and whale shark Rhincodon typus where RBCs are present without the existence of small lymphocytes. Hence, the “safe zones” for development of cancers will not be formed in the first place. FIG. 2 provides a comparison of the sizes of RBCs and lymphocytes in humans, Asian elephants, and whale shark Rhincodon typus.

Statistical data shows that overweight people are more likely to develop certain types of cancers. Applicant claims herein that, among other things, the reduction in the diameter of blood vessels in fat people is responsible for development of said types of cancers. Such reduction in the diameter of the blood vessel may be due to the fat clogging inside the vessel. It may also be a result of pressure from adjacent fat or muscle tissues outside of the vessel. The reduction in body fat reduces the fat clogging in the blood vessels. It also reduces the pressure form tissues outside of the blood vessels. Therefore, a reduction in body weight may help widen the blood vessels. As a result, WBCs will be able to enter the “safe zones” and fight cancer cells or other parasites. One way to achieve such reduction in body fat is through fasting. Another reason that fasting can be effective in treatment of chronic diseases and cancers is reported in prior arts. Prior arts show that fasting can help the treatment of some chronic diseases and cancers. According to prior arts, fasting for a long period of time decreases the insulin level in the blood which reduces the blood glucose level and triggers the formation of ketones. Unlike healthy cells, cancer cells cannot live on ketones and they need to access to the glucose for their survival. Healthy cells, however, can survive on both glucose and ketones. In addition, many types of bacteria need glucose for their survival. Therefore, according to the prior art, fasting can help the treatment of cancer or some chronic diseases by reducing the glucose level in the blood and increasing the ketones level.

Prior arts also show that anti-inflammatory drugs such as Aspirin are effective in reducing the mutation rates of the cancer cells. We claim herein that a reason why using anti-inflammatory drugs results in a reduced mutation rate is the widening of blood vessels in which the diameter of the vessel is decreased as a result of inflammation. By reducing the inflammation, the diameter of the blood vessel widens. As a result, lymphocytes will be able to enter the “safe zones” and fight the cancer cells.

This invention discloses methods to prevent or treat cancers or chronic diseases. In some example implementation of this invention, a method is implemented to widen the blood vessels to treat cancers or chronic diseases. The said widening of the blood vessels allows WBCs to enter the safe zones and fight the parasites. In some example embodiments of the invention, the said widening of the blood vessels may be achieved by administration of blood vessel widening drugs known as vasodilators. Administration of blood vessel widening drugs may be used in combination with other conventional methods for treatment of cancers or chronic diseases. For example, a vasodilator may be prescribed in combination with chemotherapy or radiotherapy for treatment of cancer. As another example, a vasodilator may be prescribed used along with antibiotics for treatment of Lyme disease.

In some example embodiments of the invention, a blood fat-reducing drug or blood clot-reducing drug is administered for treatment of cancer or chronic diseases. Administration of blood fat-reducing drugs or a blood clot-reducing drugs may be used in combination with other conventional methods for treatment of cancers or chronic diseases. For example, a blood fat-reducing drug or blood clot-reducing drug may be prescribed in combination with chemotherapy or radiotherapy for treatment of cancer. As another example, a blood fat-reducing drug or a blood clot-reducing drug may be used along with antibiotics for treatment of Lyme disease.

### REFERENCES

[1] “Impact of intermittent fasting on health and disease processes”, Mark P. Mattson, Valter D. Longo, and Michelle Harvied, Ageing Research Reviews, Vol. 39, October 2017, pp. 46-58.

[2] “These animals don't get cancer, and scientists think they could hold a cure”, David Nield, 15 Oct. 2015, https://www.sciencealert.com/these-animals-can-t-get-cancer-and-scientists-think-they-could-hold-a-cure

[3] “The animal that doesn't get cancer”, Melissa Hogenboom, 31 Oct. 2015, http.www.bbc.com/earth/story/20151031-the-animal-that-doesnt-get-cancer

[4] “Some animal species never get cancer. What can we learn from them?”Alyssa Shepard, Dec. 5, 2018, https://geneticliteracyproject.org/2018/12/05/some-animal-species-never-get-cancer-what-can-we-learn-from-them/

[5] “Atlas of Microscopic Anatomy, Section 4: Blood”, Ronald A. Bergman, Adel K. Afifi, Paul M. Heidger, Jr., https://www.anatomyatlases.org/MicroscopicAnatomy/Section04/Section04.shtml

[6] “Blood cells and serum chemistry in the world's largest fish: the whale shark Rhincodon typus”, Alistair D. M. Dove, Jill Arnold, and Tonya M. Clauss, Aquatic Biology, Vol. 9, pp. 177-183, 2010.

[7] “Hematology, Cytochemistry and Ultrastructure of Blood Cells from Asian Elephant (Elephas maximus)”, Jarernsak Salakij, Chaleow Salakij, Nual-Anong Narkkong, Suntaree Apibal, Piyawan Suthunmapinuntra, Jutamat Rattanakukuprakarn, Gavil Nunklang, and Manoch Yindee, Kasetsart J. (Nat. Sci.), Vol. 39, pp. 482-493 (2005).

1. A method to prevent or treat cancer, a chronic disease, or an infection, said method comprises of administration of a medication that widens a blood vessel, a medication that reduces the blood fat, or a medication that reduces blood clotting.

2. The method of claim 1, wherein the said method is to prevent or treat a cancer, wherein the said method further comprises of chemotherapy.

3. The method of claim 1, wherein the said method is to prevent or treat a cancer, wherein the said method further comprises of radiotherapy.

4. The method of claim 3, wherein the said method further comprises of chemotherapy.

5. The method of claim 1, wherein the said method is to prevent or treat a chronic disease or an infection, wherein the said method further comprises of administration of an antibiotic, antiviral, or an antifungal medication.

6. A method to prevent or treat cancer, a chronic disease, or an infection, said method comprises of administration of a medication that reduces the size of a white blood cell.

7. The method of claim 6, wherein the said method is to prevent or treat a cancer, wherein the said method further comprises of chemotherapy.

8. The method of claim 6, wherein the said method is to prevent or treat a cancer, wherein the said method further comprises of radiotherapy.

9. The method of claim 8, wherein the said method further comprises of chemotherapy.

10. The method of claim 6, wherein the said method is to prevent or treat a chronic disease or an infection, wherein the said method further comprises of administration of an antibiotic, antiviral, or an antifungal medication.
